(19) United States
(12) Patent Application Publication
(10) Pub. No.: US 2010/0151547 A1
(43) Pub. Date: Jun. 17, 2010

(54) System and Apparatus for Increasing Ethanol Production Efficiency

(76) Inventor: Daniel Platz, Anywhere, WI (US)

Correspondence Address: ABSOLUTE TECHNOLOGY LAW GROUP LLC, 135 W. WELLS ST., SUITE 518, MILWAUKEE, WI, 53203, US

(21) Appl. No.: 12/479,594

(22) Filed: Jun. 5, 2009

Related U.S. Application Data
(60) Provisional application No. 61/060,954, filed on Jun. 12, 2008.

(51) Int. Cl. C12P 7/06; C12M 1/00

(52) U.S. Cl. 435/161; 435/303.1

(57) ABSTRACT

The present invention is a system and apparatus for converting cellulosic material into ethanol. Cellulosic material is converted to ethanol using a self-pressurizing fermentation apparatus comprised, in various embodiments, of a pressure tank, pressure sealing means, a pressure gauge, a safety relief valve and a manual relief valve.

### CROSS-REFERENCE TO RELATED APPLICATIONS

This application claims priority to U.S. Provisional Application No. 61/060,954 filed on Jun. 12, 2008.

### FIELD OF INVENTION

The present invention relates to the field of ethanol production, and more specifically to a system and apparatus for producing ethanol from cellulosic material.

### BRIEF DESCRIPTION OF THE DRAWINGS

FIG. 1 illustrates an exemplary embodiment of a self-pressurizing fermentation apparatus.

FIG. 2 illustrates how a method is performed using an exemplary embodiment of a self-pressurizing fermentation apparatus.

FIG. 3 illustrates the steps of a method to convert cellulosic material to ethanol.

### GLOSSARY

As used herein, the term “ethanol” means a volatile, flammable, colorless liquid. Ethanol is also called ethyl alcohol, pure alcohol, grain alcohol, or drinking alcohol. Ethanol has widespread use as a solvent of substances intended for human contact or consumption, including scents, flavorings, colorings, and medicines. Ethanol is also an essential solvent and a feedstock for the synthesis of other products. Most importantly, ethanol is used for heat, light, and as a fuel for internal combustion engines.

As used herein, the term “cellulosic material” means any material containing cellulose, a polysaccharide consisting of a linear chain of, several hundred to over ten thousand linked D-glucose units. Examples of cellulosic materials include but are not limited to wood, wood pulp, sawdust, paper, cotton, grasses, plants, algae, manufactured materials and any other material containing cellulose.

As used herein, the term “pressure gauge” means any instrument that is capable of measuring pressure within a pressure tank.

As used herein, the term “safety relief valve” means a safety device that automatically relieves static pressure in case of overpressure in a vessel or piping.

As used herein, the term “manual relief valve” means a device that allows an individual to manually relieve pressure in a vessel or piping.

As used herein, the term “pressure tank” means any closed container or vessel or a plurality of closed containers or vessels acting as a system that is capable of being sealed to impede gasses from escaping during a fermentation process and/or which is capable of maintaining a pressure different from an ambient pressure.

As used herein, the term “pressure sealing means” is a means of securing a lid or lid assembly to a pressure tank so as not to allow gas, vapors, liquids or any combination thereof to escape said pressure tank. Pressure sealing means may include but are not limited to clamps, bolts, rubber seals, wing nuts, sealing compounds known in the art, specially contoured or engineered lids or other components to create a seal, gaskets, polymers and any other substance known the art to create a seal.

As used herein, the term “pre-calibrated pressurization level” is a level at which a safety relief valve known in the art is callibrated or designed to automatically relieve pressure when the pressure reaches the pre-determined level.

As used herein, the term “remote monitoring device” is any device known in the art which allows the diagnostics of the self-pressurizing fermentation apparatus and system and apparatus for increasing ethanol production efficiency to be monitorized remotely. For example, remote monitoring device will alert the user when the pressure of self-pressurizing fermenation apparatus reaches a pre-determined level and the case of an equipment failure or system malfunction. In other embodiments, remote monitoring device can be programmed' to alert the user when other pre-determined criteria are reached.

### BACKGROUND

The United States currently produces about 5 billion gallons a year of traditional ethanol. Demand has risen as the result of high oil prices and low domestic crude production, pushing the United States to set a goal of making 60 billion gallons per year of ethanol by 2030.

Ethanol is a viable, commercial, and relatively clean fuel substitute or additive. As of 2007, ethanol is produced mainly from sugars or starches, i.e., starch ethanol. Starch ethanol is easily obtained from the fermentation of grain or other substances containing sugar or starches and is commonly made from common crops such sugar cane and corn. These crops have alternative uses as food sources for human populations.

Non-edible cellulosic may also be used to produce ethanol; thus creating fuel without affecting human food supplies. Cellulosic ethanol has other advantages over ethanol produced from starches. Studies have shown that one of the benefits, of cellulosic ethanol is that it reduces greenhouse gas emissions by 85% over reformulated gasoline. In contrast, ethanol made from corn (starch ethanol) may not reduce greenhouse gas emissions depending on how the starch-based feedstock is produced. In addition, the production of starch ethanol requires natural gas to provide energy for the process reducing the total amount of energy saved by using starch ethanol instead of natural gas.

Another advantage to cellulosic ethanol is that cellulosic material cannot be digested by humans; therefore, the production of cellulosic ethanol does not compete with the production of food for human consumption (other than conversion of land from food production to cellulose production) as does starch ethanol. In addition, cellulosic material can be grown in all parts of the world and the entire plant can be used when producing ethanol. Thus, the price per ton of cellulosic raw material is much cheaper and less volatile (e.g., corn futures are traded on commodity exchanges) than grains for fruits.

In addition, cellulosic material is found in waste material, such as waste paper, waste pulp from paper factories, spent cellulose from paper recycling plants, agricultural waste, refuse from food processing plants, municipal solid waste residual fluff (e.g., paper scraps, lawn wastes, newsprint and cardboard, packaging wastes, food and food wastes), and wood products.

There are many processes known in the art for producing ethanol from cellulosic material. These processes are thermally inefficient and require the use of acids and other costly substances which decrease the net energy and cost savings of using cellulosic ethanol.

One, example of a process for producing cellulosic ethanol known in the art is U.S. Pat. No. 5,135,861 (Pavilon '861). Pavilon '861 teaches a method of producing ethanol from cellulosic material. A slurry of cellulosic material is first hydrolyzed in a fuel fired hydrolysis heater. Solids are separated from the hydrolyzed effluent and dried using the hydrolysis heater. The effluent is fermented and subsequently distilled.

It is desirable to have a method for producing cellulosic ethanol that does not require the use of an external heat source for hydrolysis or fermentation to occur.

Another example known in the art is taught by U.S. Pat. No. 5,620,877 (Farone et al. '877). Farone et al. '877 teaches a method of fermenting a mixture of sugars resulting from the acid hydrolysis of cellulosic material. The sugar solution is mixed with a microbial organism known to produce a useful fermentation product, and the fermentation process is allowed to proceed for 3 to 5 days, during and after which the fermentation products are removed and purified.

While the process taught by Farone et al. '877 is more energy efficient than the process taught by Pavilon '861 because it does not use an external heat source, it requires the addition of acid for hydrolysis. It is desirable to have a method for producing cellulosic ethanol that does not first require treating the cellulosic material with an acid or the addition of any acid during the fermentation process. It is further desirable to utilize the acid created during fermentation.

Another example known in the art is taught by U.S. Pat. No. 5,879,637 (Titmas '637). Titmas '637 teaches a method for producing cellulosic ethanol using a gravity pressure vessel. Cellulosic material is added to a gravity pressure vessel where it is subject to the proper conditions for carrying out acid hydrolysis. The liquid stream is removed and delivered to a post treatment clarifier where heavy refractory cellulose and inert precipitates are removed. The clarified aqueous sugar solution, which still contains residual particles of unreacted cellulose, is delivered from the separator to a fermentation apparatus where fermentation is carried out.

Titmas '637 also teaches an apparatus for carrying out necessary reactions to produce ethanol from cellulosic material. The apparatus disclosed by Titmas '637 requires various pumping devices, conduits and storage areas in order to move material between the various stages of the system and maintain the process in continuous operation.

It is desirable to have a system and apparatus which allows hydrolysis and fermentation to be carried out simultaneously in a single vessel.

It is further desirable to have a system and apparatus that allows for production of ethanol from cellulosic material which uses by-products of the reaction to further facilitate the reaction.

It is further desirable to have a system and apparatus that allows for production of ethanol from cellulosic material which results in a negligible amount of waste.

### SUMMARY OF THE INVENTION

The present invention is a system and apparatus for converting cellulosic material into ethanol.

### DETAILED DESCRIPTION OF INVENTION

For the purpose of promoting an understanding of the present invention, references are made in the text to exemplary embodiments of a system and apparatus for producing ethanol from cellulosic material, only some of which are described herein. It should be understood that no limitations on the scope of the invention are intended by describing these exemplary embodiments. One of ordinary skill in the art will readily appreciate that alternate but functionally equivalent ratios, volumes, masses, and incubation times may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

It should be understood that the drawings are not necessarily to scale; instead, emphasis has been placed upon illustrating the principles of the invention. In addition, in the embodiments depicted herein, like reference numerals in the various drawings refer to identical or near identical structural elements.

Moreover, the terms “substantially” or “approximately” as used herein may be applied to modify any quantitative representation that could permissibly vary without resulting in a change in the basic function to which it is related.

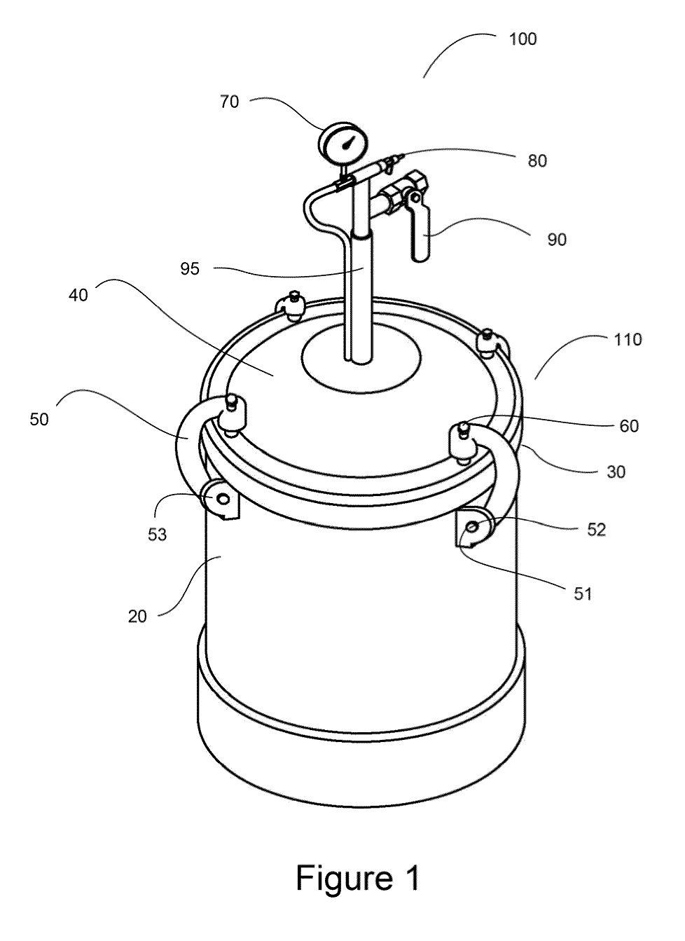

FIG. 1 is an exemplary embodiment of self-pressurizing fermentation apparatus 100 used to create ethanol from cellulosic material. Pressure tank 10 consists of vessel 20, lid assembly 40 and lid gasket 30.

In the embodiment shown, pressure tank 10 a pressure tank capable of withstanding of pressures from a range 5 to 2000 psi. In the embodiment shown, pressure tank 10 is comprised of a metallic or plastic outer surface and may or may not include a reinforcing liner (not shown) that is moisture and/or heat resistant and capable of withstanding high temperatures and pressures.

FIG. 1 further illustrates pressure sealing means, which in the embodiment shown are multiple clamps 50 secured to vessel 20 by passing hinge pins 52 through apertures 51 in protuberances 53 on tank shell assembly 20 and then through apertures 56 (not shown) in the bottom of clamps 50. In the embodiment shown, retaining rings 58 (not shown) are then passed through apertures 57 (not shown) in the non-head end of hinge pins 52 securing the bottom of clamps 50 to tank shell assembly 20. Bolts 60 are threaded through apertures 62 (not shown) in the top flat surface of clamps 50. When bolts 60 are tightened, they apply downward pressure on lid assembly 40 ensuring that a seal is created between lid assembly 40 and tank shell assembly 20. Other embodiments may include more or fewer clamps and may omit or include bolts, hinge pins, etc. Pressure sealing means may be any structural configuration known in the art to impede the escape of gasses, vapors, liquids and any combination thereof from vessel 20.

In alternate embodiments, pressure sealing means 50 may include or be comprised of clamps, bolts, rubber seals, wing nuts, sealing compounds known in the art, specially contoured or engineered lids or other components to create a seal, gaskets, polymers and any other substance known the art to create a seal.

In the embodiment shown in FIG. 1, self-pressurizing fermentation apparatus 100 further includes pressure gauge 70, safety relief valve 80, and manual relief valve 90. In the embodiment shown, pressure gauge 70, safety relief valve 80, and manual relief valve 90 are connected to lid assembly 40 via brass piping 95, but may be attached or using other types of piping or by any means of permanent or non-permanent attachment known in the art. For example, in other embodiments, pressure gauge 70 and safety relief valve 80 may be selectively attached, replaced or repositioned. Still other embodiments may include multiple pressure gauges 70 and safety relief valves 80, and operation of pressure gauges 70 and safety relief valves 80 may be computerized, automated, manual or any combination thereof.

In alternate embodiments, self-pressurizing fermentation apparatus 100 further includes a remote monitoring device, which allows self-pressurizing fermentation apparatus 100 to be monitored remotely.

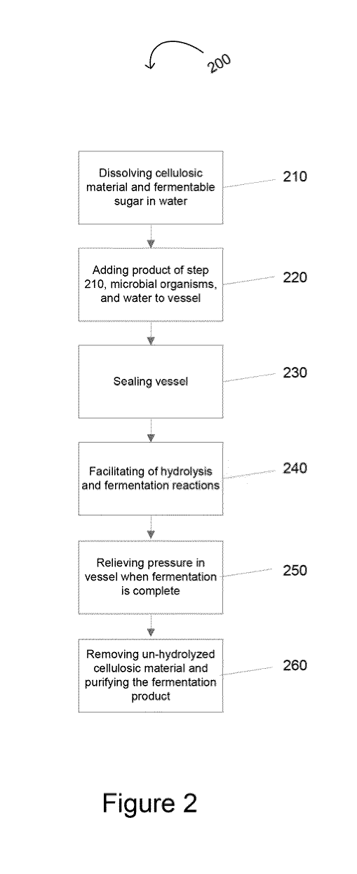

FIG. 2 illustrates an exemplary method for converting cellulosic material to ethanol 200 using self-pressurizing fermentation apparatus 100. Other methods using self-pressurizing fermentation apparatus 100 may include more, fewer or equivalent steps.

In Step 210, cellulosic material and fermentable sugar are dissolved in water. In various embodiments of method for converting cellulosic material to ethanol 200, Step 210 may include dissolving 25 grams of cellulosic material and 75 grams of fermentable sugar per liter of water. In an exemplary embodiment, sawdust and sucrose are dissolved in water. In other embodiments, another type of cellulosic material, fermentable sugar; or both are used. In addition, in other embodiments, the ratios of cellulosic material and fermentable sugar may vary.

In Step 220, the resulting slurry of cellulosic material and fermentable sugar is added to vessel 20 (not shown) of self-pressurizing fermentation apparatus 100. Water and a microbial organism known to produce a useful fermentation product from fermentable sugar are then added to vessel 20 (not shown). In an exemplary embodiment, Saccharomyces cerevisiae, a strain of yeast, is used. In other embodiments, a strain of yeast other than Saccharomyces cerevisiae is used, or a bacterial strain capable of fermenting sugar. In an exemplary embodiment, dry ice (solid phase of carbon dioxide) is also added to vessel 20 (not shown) to accelerate the removal of oxygen increasing the concentration of carbon dioxide. In other embodiments, a source of carbon dioxide other than dry ice may be used, such as gaseous carbon dioxide or nitrogen.

In Step 230, vessel 20 (not shown) is tightly sealed in order to ensure that the apparatus's internal pressure can build to an optimal level. The increase in pressure occurs because the fermentation process breaks down the fermentable sugar into ethanol and carbon dioxide molecules increasing the number of moles of fermentation product.

In Step 240, carbon dioxide created during fermentation combines with water under pressure to form carbonic acid which has the capability of breaking the oxygen linkage between the sugar molecules which make up cellulose. The resulting hydrolyzed sugar molecules are then fermented creating a continuous process of fermentation and hydrolysis. Hydrolysis and fermentation continue without the need to adjust manually, the pressure inside vessel 20 (not shown). During hydrolysis and fermentation, the pressure inside vessel 20 is regulated by safety relief valve 80.

In Step 250, fermentation is complete and the pressure is relieved from within vessel 20. In an exemplary embodiment, pressure is released by opening manual relief valve 90. Lid 40 is removed from vessel 20. In an exemplary embodiment, the duration for fermentation is 48 to 72 hours. In other embodiments, fermentation is allowed to continue for a lesser or greater number of hours.

In Step 260, any un-hydrolyzed cellulosic material is removed from vessel 20 and the fermentation product is purified.

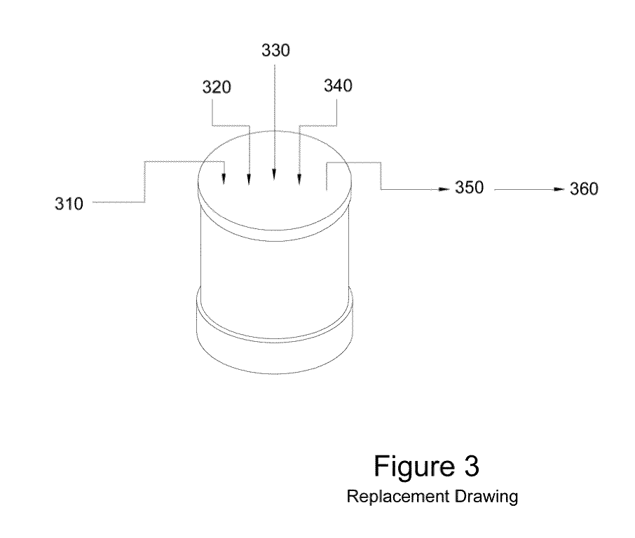

FIG. 3 illustrates the exemplary use of a self-pressurizing fermentation apparatus 100 as described in FIG. 1 to create ethanol from cellulosic material. As illustrated in FIG. 2, cellulosic material and fermentable sugar are dissolved in water (not shown) and the resulting cellulose and sugar slurry 310 is added to self-pressurizing fermentation apparatus 100. A microbial organism known to produce a useful fermentation product from fermentable sugar 320, water 330, and a carbon dioxide source 340 (e.g., dry ice) are added to vessel 20. In the embodiment shown, microbial organism known to produce a useful fermentation product from fermentable sugar 320 is yeast, specifically Saccharomyces cerevisiae. In other embodiments, microbial organism known to produce a useful fermentation product from fermentable sugar 320 is a strain of yeast other than Saccharomyces cerevisiae, or a bacterial strain capable of fermenting sugar. After hydrolysis and fermentation are complete, fermentation product 350, which contains ethanol 360, water 330 and microbial organism known to produce a useful fermentation product from fermentable sugar 320 is removed from self-pressurizing fermentation apparatus 100 and purified resulting in ethanol 360.

Self-pressurizing fermentation apparatus 100 is sealed using pressure sealing means 50.

What is claimed is:

1. A self-pressurizing fermentation apparatus for creating ethanol from cellulosic material comprised of:
at least one pressure tank;
at least one pressure sealing means;
at least one pressure gauge;
at least one safety relief valve to prevent pressurization above a pre-calibrated pressurization level; and
at least one manual relief valve.

2. The self-pressurizing fermentation apparatus of claim 1 which further includes multiple interconnected tanks.

3. The self-pressurizing fermentation apparatus of claim 1 wherein said pressure sealing means is selected from a group consisting clamps, bolts, rubber seals, wing nuts, sealing compounds and specially contoured lids.

4. The self-pressurizing fermentation apparatus of claim 1 wherein said pressure tank includes a reinforcing liner.

5. The self-pressurizing fermentation apparatus of claim 1 wherein said safety relief valve is configured with software to relieve pressure when pressure inside said pressure tank reaches said pre-calibrated pressurization level.

6. The self-pressurizing fermentation apparatus of claim 1 which further includes at least one remote monitoring device.

7. A self-pressurizing fermentation apparatus for creating ethanol from cellulosic material comprised of:
at least one pressure tank capable of withstanding pressures between 5 and 2000 psi;
at least one pressure sealing means;
at least one pressure gauge;
at least one safety relief valve to prevent pressurization above a pre-calibrated pressurization level; and
at least one manual relief valve.

8. The self-pressurizing fermentation apparatus of claim 7 which further includes multiple interconnected tanks.

9. The self-pressurizing fermentation apparatus of claim 7 wherein said pressure sealing means is selected from a group consisting clamps, bolts, rubber seals, wing nuts, sealing compounds and specially contoured lids.

10. The self-pressurizing fermentation apparatus of claim 7 wherein said pressure tank includes a reinforcing liner.

11. The self-pressurizing fermentation apparatus of claim 7 wherein said safety relief valve is configured with software to relieve pressure when pressure inside said pressure tank reaches said pre-calibrated pressurization level.

12. The self-pressurizing fermentation apparatus of claim 7 which further includes at least one remote monitoring device.

13. A method of converting cellulosic material into ethanol utilizing a self-pressurizing fermentation apparatus comprised of the steps of:
dissolving cellulosic material and fermentable sugar in water;
adding said dissolved cellulosic material and fermentable sugar to a self-pressurizing fermentation apparatus;
adding a microbial organism known to produce a useful fermentation product from fermentable sugar to said self-pressurizing fermentation apparatus;
adding water to said self-pressurizing fermentation apparatus;
sealing said self-pressurizing fermentation apparatus;
allowing hydrolysis and fermentation processes to occur;
relieving pressure from said self-pressurizing fermentation apparatus;
collecting fermentation product from said self-pressurizing fermentation apparatus; and
purifying said fermentation products.

14. The method of claim 13 wherein the said microbial organism known to produce a useful fermentation product from fermentable sugar is Saccharomyces cerevisiae.

15. The method of claim 13 which further includes the step of monitoring the pressure of said self-pressurizing fermentation apparatus using a safety valve.

16. The method of claim 13 which further includes the step of monitoring said self-pressurizing fermentation apparatus remotely.

17. The method of claim 13 which further includes the step of measuring the pressure of said self-pressurizing fermentation apparatus using a pressure gauge.

18. The method of claim 13 which further includes the step of adding a carbon dioxide source to said self-pressurizing fermentation apparatus.

19. The method of claim 18 wherein said carbon dioxide source is dry ice.
